(19) United States
(12) Patent Application Publication
(10) Pub. No.: US 2008/0303885 A1
(43) Pub. Date: Dec. 11, 2008

(54) INKJET INK COMPOSITION, AND INK SET, CARTRIDGE, AND INKJET RECORDING APPARATUS USING THE SAME

(75) Inventor: Yeon-kyoung JUNG, Suwon-si (KR)

Correspondence Address: STANZIONE & KIM, LLP, 919 18TH STREET, N.W., SUITE 440, WASHINGTON, DC, 20006, US

(73) Assignee: Samsung Electronics Co., Ltd., Suwon-si (KR)

(21) Appl. No.: 12/024,277

(22) Filed: Feb. 1, 2008

(30) Foreign Application Priority Data
Jun. 8, 2007 (KR) 2007-56055

(51) Int. Cl. C09D 11/02; C07C 255/00; B41J 2/175

(52) U.S. Cl. 347/100; 106/31.13; 106/31.75; 558/303; 347/86

(57) ABSTRACT

An inkjet ink composition includes a colorant, water, and a dicyanobenzene-based compound, and an ink set including the same.

### CROSS-REFERENCE TO RELATED APPLICATIONS

This application claims priority under 35 U.S.C. §119(a) from Korean Patent Application No. 10-2007-0056055, filed on Jun. 8, 2007, in the Korean Intellectual Property Office, the disclosure of which is incorporated herein in its entirety by reference.

### BACKGROUND OF THE INVENTION

1. Field of the Invention

The present general inventive concept relates to an inkjet ink composition and an ink set including the same. More particularly, the present general inventive concept relates to an inkjet ink composition with improved water repellency, including a colorant, water, and a dicyanobenzene-based compound, and an ink set including the same.

2. Description of the Related Art

Generally, inkjet recording apparatuses realize images on media by ejecting ink droplets onto the media using fine nozzles. Inkjet ink must satisfy various requirements, such as good drying characteristics on media, no image blurring, and uniform images, regardless of the type of media. Moreover, when various colors are printed, color mixing must not occur at boundaries between the colors, and image durability characteristics, such as water repellency, light fastness, and anti-frictional property, must be excellent.

In order to satisfy the above requirements, various ink compositions have been proposed. In particular, in order to improve the durability (particularly, water repellency) of images, various attempts have been made.

For example, in U.S. Pat. No. 5,026,425, in order to improve the water repellency of an ink composition, sodium cations of Direct Black 168 dye are exchanged with ammonium, polyfunctional and volatile amine cations by ion exchange. In U.S. Pat. No. 5,026,426, the water repellency of an ink composition is improved by adding a low molecular weight nitrile compound, in particular, an amine functional group-containing nitrile compound. U.S. Pat. No. 5,958,120 discloses an ink composition with improved water repellency, including a vinyl sulfate end functional group-containing reactive dye, N′-bis(3-aminopropyl)-1,2-ethylenediamine, imidazole, phosphoric acid or phosphite salt, tripropylene glycol monomethyl ether, a Zn²⁺ salt, etc.

In spite of the above-described various ink compositions, in order to satisfy requirements, such as a high image concentration on media, good durability, and stability against nozzle clogging, it is still necessary to improve ink compositions.

### SUMMARY OF THE INVENTION

The present general inventive concept provides an inkjet ink composition which has a minimal degree of blurring of printed images with water on a general paper and which causes no nozzle clogging.

The present general inventive concept also provides an ink set including the ink composition.

Additional aspects and utilities of the present general inventive concept will be set forth in part in the description which follows and, in part, will be obvious from the description, or may be learned by practice of the general inventive concept.

The foregoing and/or other aspects and utilities of the present general inventive concept may be achieved by providing an inkjet ink composition including a colorant, a solvent, and a compound represented by Formula 1 below:

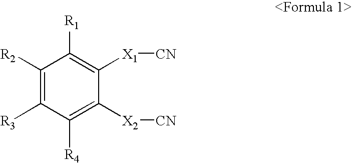

wherein, R₁, R₂, R₃, and R₄ may independently be one of a hydrogen atom, a nitro group, an amino group, a hydroxy group, halogen, a cyano group, a carboxyl group, a substituted or unsubstituted C1-C20 alkyl group, a substituted or unsubstituted C2-C20 alkenyl group, a substituted or unsubstituted C2-C20 alkynyl group, a substituted or unsubstituted C1-C20 alkoxy group, a substituted or unsubstituted C2-C20 alkoxycarbonyl group, a substituted or unsubstituted C6-C30 aryl group, a substituted or unsubstituted C1-C20 heteroalkyl group, a substituted or unsubstituted C3-C20 heteroaryl group, a substituted or unsubstituted C2-C20 alkylcarbonyl group, a substituted or unsubstituted C7-C30 arylcarbonyl group, and a substituted or unsubstituted C4-C30 heteroarylcarbonyl group, and at least one of R₁, R₂, R₃, and R₄ includes at least one functional group selected from a group consisting of an amino group, a nitro group, and a hydroxy group, and X₁ and X₂ may each independently be one of a bond, C1-C20 alkylene, C2-C20 alkenylene, C2-C20 alkynylene, C6-C30 arylene, C1-C20 heteroalkylene, and C3-C30 heteroarylene.

In the compound of Formula 1, R₁, R₂, R₃, and R₄ may be each independently be one of a hydrogen atom, a nitro group, an amino group, a hydroxy group, a C1-C20 alkyl group, a C2-C20 alkenyl group, and a C2-C20 alkynyl group, at least one hydrogen atom of the alkyl group, the alkenyl group, or the alkynyl group may be substituted by one of a nitro group, an amino group, and a hydroxy group, and at least one of R₁, R₂, R₃, and R₄ may include at least one functional group selected from a group consisting of a nitro group, an amino group, and a hydroxy group.

In the compound of Formula 1, each of X₁ and X₂ may be a bond.

The compound of Formula 1 may be a compound represented by Formula 2 below:

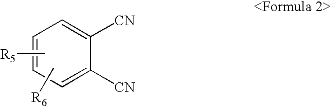

wherein, R₅ and R₆ may each independently be one of a nitro group, an amino group, a hydroxy group, a C1-C20 alkyl group, a C2-C20 alkenyl group, and a C2-C20 alkynyl group, and at least one hydrogen atom of the alkyl group, the alkenyl group, or the alkynyl group may be substituted by one of a nitro group, an amino group, and a hydroxy group.

The compound of Formula 1 may be one of 4-aminophthalonitrile, 2,3-dicyanohydroquinone, 4-nitrophthalonitrile, and the like.

A content of the compound of Formula 1 may be 0.1 to 10 parts by weight, preferably 1 to 7 parts by weight, based on 100 parts by weight of the inkjet ink composition.

The colorant may be one of a dye, a pigment, a self-dispersible pigment, and a mixture thereof.

A content of the colorant may be 0.1 to 15 parts by weight based on 100 parts by weight of the inkjet ink composition.

The solvent may be one of an aqueous solvent, an organic solvent, and a mixture thereof.

A content of the solvent may be 70 to 90 parts by weight based on 100 parts by weight of the inkjet ink composition.

The organic solvent may be at least one of an alcohol-based solvent, a ketone-based solvent, an ester-based solvent, a polyhydric alcohol-based solvent, a nitrogen-containing solvent, and a sulfur-containing compound of dimethylsulfoxide, tetramethylsulfone, and thioglycol.

The solvent may include 100 parts by weight of an aqueous solvent and 0.1 to 130 parts by weight of an organic solvent.

The inkjet ink composition may have a surface tension of 15 to 70 dyne/cm at 20° C.

The inkjet ink composition may have a viscosity of 1.5 to 20 cps.

The foregoing and/or other aspects and utilities of the present general inventive concept may also be achieved by providing a polychromatic ink set including two or more kinds of inkjet ink compositions, each inkjet ink composition including a colorant, a solvent, and a compound represented by Formula 3 below:

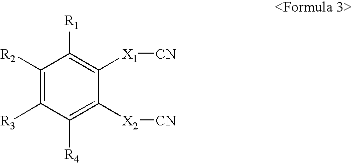

wherein R1, R2, R3, and R4 are each independently one of a hydrogen atom, a nitro group, an amino group, a hydroxy group, halogen, a cyano group, a carboxyl group, a substituted or unsubstituted C1-C20 alkyl group, a substituted or unsubstituted C2-C20 alkenyl group, a substituted or unsubstituted C2-C20 alkynyl group, a substituted or unsubstituted C1-C20 alkoxy group, a substituted or unsubstituted C2-C20 alkoxycarbonyl group, a substituted or unsubstituted C6-C30 aryl group, a substituted or unsubstituted C1-C20 heteroalkyl group, a substituted or unsubstituted C3-C20 heteroaryl group, a substituted or unsubstituted C2-C20 alkylcarbonyl group, a substituted or unsubstituted C7-C30 arylcarbonyl group, and a substituted or unsubstituted C4-C30 heteroarylcarbonyl group, and at least one of R1, R2, R3, and R4 comprises at least one functional group selected from a group consisting of an amino group, a nitro group, and a hydroxy group, and X1 and X2 are each independently one of a bond, C1-C20 alkylene, C2-C20 alkenylene, C2-C20 alkynylene, C6-C30 arylene, C1-C20 heteroalkylene, and C3-C30 heteroarylene.

The foregoing and/or other aspects and utilities of the present general inventive concept may also be achieved by providing a cartridge for an inkjet recording apparatus, including the polychromatic ink set.

The foregoing and/or other aspects and utilities of the present general inventive concept may also be achieved by providing an inkjet recording apparatus including the cartridge.

The inkjet recording apparatus may include a thermal head.

The inkjet recording apparatus may include an array head including 10,000 or more nozzles.

The foregoing and/or other aspects and utilities of the present general inventive concept may also be achieved by providing an inkjet ink composition including a colorant, a solvent, and a crosslinker to link the colorant to a printing medium.

The crosslinker may include a nitrile group to bind with the colorant, and one of a nitro group, amino group, and hydroxy group to bind with the printing medium.

The crosslinker can be represented by Formula 4 below:

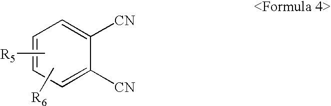

R5 and R6 may each independently be one of a nitro group, an amino group, a hydroxy group, a C1-C20 alkyl group, a C2-C20 alkenyl group, and a C2-C20 alkynyl group, and at least one hydrogen atom of the alkyl group, the alkenyl group, or the alkynyl group is substituted by one of a nitro group, an amino group, and a hydroxy group.

The crosslinker may be one of 4-aminophthalonitrile, 2,3-dicyanohydroquinone, and 4-nitrophthalonitrile.

The foregoing and/or other aspects and utilities of the present general inventive concept may also be achieved by providing a crosslinker usable in inkjet ink composition to chemically link a colorant in the ink composition to a printing medium, the crosslinker including a nitrile group to bind with the colorant, and one of a nitro group, amino group, and hydroxy group to bind with the printing medium.

The crosslinker may be a dicyanobenzene-based compound.

### BRIEF DESCRIPTION OF THE DRAWINGS

These and/or other aspects and utilities of the present general inventive concept will become apparent and more readily appreciated from the following description of the embodiments, taken in conjunction with the accompanying drawings of which:

FIG. 1 is a perspective view illustrating an inkjet recording apparatus including an ink cartridge including an ink composition according to the present general inventive concept; and

FIG. 2 is a sectional view illustrating an ink cartridge including an ink composition according to the present general inventive concept.

### DETAILED DESCRIPTION OF THE PREFERRED EMBODIMENTS

Reference will now be made in detail to the embodiments of the present general inventive concept, examples of which are illustrated in the accompanying drawings, wherein like reference numerals refer to the like elements throughout. The embodiments are described below in order to explain the present general inventive concept by referring to the figures.

An inkjet ink composition according to the present general inventive concept may include a colorant, a solvent, and a compound represented by Formula 1 below. The compound of Formula 1 acts as a crosslinker, linking a paper and the colorant, thus improving durability (particularly, water repellency) of images:

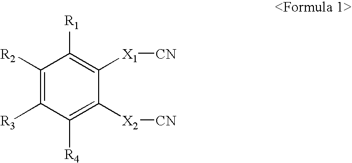

In Formula 1, R₁, R₂, R₃, and R₄ may be the same or different and each may be a hydrogen atom, a nitro group, an amino group, a hydroxy group, halogen, a cyano group, a carboxyl group, a substituted or unsubstituted C1-C20 alkyl group, a substituted or unsubstituted C2-C20 alkenyl group, a substituted or unsubstituted C2-C20 alkynyl group, a substituted or unsubstituted C1-C20 alkoxy group, a substituted or unsubstituted C2-C20 alkoxycarbonyl group, a substituted or unsubstituted C6-C30 aryl group, a substituted or unsubstituted C1-C20 heteroalkyl group, a substituted or unsubstituted C3-C20 heteroaryl group, a substituted or unsubstituted C2-C20 alkylcarbonyl group, a substituted or unsubstituted C7-C30 arylcarbonyl group, or a substituted or unsubstituted C4-C30 heteroarylcarbonyl group, and at least one of R₁, R₂, R₃, and R₄ includes at least one functional group selected from the group consisting of an amino group, a nitro group, and a hydroxy group.

Similarly, X₁ and X₂ can be the same or different, and each may be a bond, C1-C20 alkylene, C2-C20 alkenylene, C2-C20 alkynylene, C6-C30 arylene, C1-C20 heteroalkylene, or C3-C30 heteroarylene.

As described above, the inkjet ink composition according to the present general inventive concept includes a compound of Formula 1. At least one of R₁, R₂, R₃, and R₄ in the compound of Formula 1 includes a nitro group, an amino group, or a hydroxy group, and thus, can form a hydrogen bond with cellulose present on a paper. Moreover, a nitrile group present in the compound of Formula 1 can bind with the colorant. Therefore, the compound of Formula 1 can allow a strong binding between a paper and the colorant by chemical bonds, such as hydrogen bonds, instead of simple physical bonds, such as adsorption. Such a strong binding between the colorant and the paper improves durability (particularly, water repellency) of images, thereby giving a minimal degree of blurring of printed images with water on a general paper.

Thus, the compound of Formula 1 included in the inkjet ink composition of the present general inventive concept includes a nitrile group capable of binding with the colorant, and a nitro, amino, or hydroxy group capable of binding with a paper.

In the compound of Formula 1, R₁, R₂, R₃, and R₄ may be each independently a hydrogen atom, a nitro group, an amino group, a hydroxy group, a C1-C20 alkyl group, a C2-C20 alkenyl group, or a C2-C20 alkynyl group, at least one hydrogen atom of the alkyl group, the alkenyl group, or the alkynyl group may be substituted by a nitro group, an amino group, or a hydroxy group, and at least one of R₁, R₂, R₃, and R₄ may include at least one functional group selected from the group consisting of a nitro group, an amino group, and a hydroxy group. Each of X₁ and X₂ may be a bond.

For example, the compound of Formula 1 may be a compound of Formula 2 below:

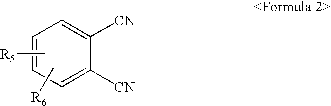

Similarly, in Formula 2, R₅ and R₆ can each independently be a nitro group, an amino group, a hydroxy group, a C1-C20 alkyl group, a C2-C20 alkenyl group, or a C2-C20 alkynyl group, and at least one hydrogen atom of the alkyl group, the alkenyl group, or the alkynyl group may be substituted by a nitro group, an amino group, or a hydroxy group.

Particularly, the compound of Formula 1 can be 4-aminophthalonitrile, 2,3-dicyanohydroquinone, or 4-nitrophthalonitrile.

Substituents used in compounds according to the present general inventive concept can be defined as follows.

The term “alkyl” refers to a straight or branched, saturated monovalent hydrocarbon moiety having 1-20, preferably 1-10, more preferably 1-6, carbon atoms. The alkyl group may be optionally substituted by one or more halogen substituents. Examples of the alkyl group include methyl, ethyl, propyl, 2-propyl, n-butyl, iso-butyl, tert-butyl, pentyl, hexyl, dodecyl, fluoromethyl, difluoromethyl, trifluoromethyl, chloromethyl, dichloromethyl, trichloromethyl, iodomethyl, and bromomethyl.

The term “aryl” refers to a monovalent monocyclic, bicyclic, or tricyclic aromatic hydrocarbon moiety having 6-30, preferably 6-18, cyclic atoms. The aryl group may be optionally substituted by at least one halogen substituent.

The term “alkenyl” refers to a straight or branched unsaturated hydrocarbon chain radical having 2-20, preferably 2-10, more preferably 2-6, carbon atoms. The straight or branched hydrocarbon chain radical consists of carbon and hydrogen atoms, and includes at least one double bond. For example, the alkenyl group may be ethyl, prop-1-enyl, but-1-enyl, pent-1-enyl, penta-1,4-diethyl, etc.

The term “alkynyl” refers to a straight or branched unsaturated hydrocarbon chain radical having 2-20, preferably 2-10, more preferably 2-6, carbon atoms. The straight or branched hydrocarbon chain radical consists of carbon and hydrogen atoms, and includes at least one triple bond. For example, the alkynyl group may be ethynyl, prop-1-ynyl, but-1-ynyl, pent-1-ynyl, pent-3-enyl, etc.

The term “alkoxy” refers to a group having the formula alkyl-O— where alkyl is as defined above.

The term “heteroalkyl” refers to an alkyl group as defined above in which at least one carbon atom in the main chain is substituted by at least one heteroatom, e.g., nitrogen, sulfur, oxygen, or phosphorus.

The term “heteroaryl” refers to an aryl group as defined above in which at least one carbon atom in the ring is substituted by at least one heteroatom, e.g., nitrogen, sulfur, oxygen, or phosphorus.

The terms “alkylene”, “alkenylene”, and “alkynylene” refer to divalent alkyl, alkenyl, and alkynyl groups, respectively.

In the inkjet ink composition of the present general inventive concept, the content of the compound of Formula 1 may be 0.1 to 10 parts by weight, preferably 1 to 7 parts by weight, based on 100 parts by weight of the inkjet ink composition. If the content of the compound of Formula 1 is less than 0.1 parts by weight, improvement in durability of printed images may be insignificant. On the other hand, if the content of the compound of Formula 1 exceeds 10 parts by weight, problems such as clogging may occur.

The inkjet ink composition of the present general inventive concept includes a colorant and a solvent, in addition to the compound of Formula 1. The choice of colorant used is not limited provided that it can be used in an inkjet ink composition. Thus, the colorant may be a dye that can be dissolved or dispersed in a solvent (e.g., water), a pigment that can be stably dispersed in water in the presence of a dispersing agent, a self-dispersible pigment that can be stably dispersed in water in the absence of a dispersing agent, or a mixture thereof.

Example of a dye that can be used as the colorant include direct dyes, acid dyes, foodstuff dyes, basic dyes, reactive dyes, disperse dyes, vat dyes, soluble vat dyes, reactive disperse dyes, etc., and examples of a pigment that can be used as the colorant include carbon black, graphite, vitreous carbon, activated charcoal, activated carbon, anthraquinone, phthalocyanine blue, phthalocyanine green, diazos, monoazos, pyranthrones, perylene, quinacridone, indigoid pigments, self-dispersible pigments, etc. However, the present general inventive concept is not limited thereto, and various other colorants may be used.

The content of the colorant may be 1 to 15 parts by weight, preferably 1 to 10 parts by weight, based on 100 parts by weight of the ink composition. If the content of the colorant is less than 1 part by weight, sufficient coloration may not be achieved. On the other hand, if the content of the colorant exceeds 15 parts by weight, economical advantages may not be achieved due to an excess amount of the colorant.

The solvent included in the ink composition of the present general inventive concept may be an aqueous medium. The aqueous medium may be water alone or in combination with at least one organic solvent. The content of the solvent may be 70 to 90 parts by weight based on the total weight of the ink composition. If the content of the solvent is less than 70 parts by weight, the viscosity of the ink composition may be excessively increased, thereby lowering ejection performance. If the content of the solvent exceeds 90 parts by weight, the viscosity of the ink composition may be excessively decreased.

The organic solvent included in the solvent may be one of primary alcohol-based solvents, ketone-based solvents, ester-based solvents, polyhydric alcohol-based solvents, polyhydric alcohol-based derivative solvents, nitrogen-containing solvents, and sulfur-containing compounds (dimethylsulfoxide, tetramethylsulfone, and thioglycol). For example, the primary alcohol-based solvents can adjust the surface tension of ink, thereby improving an ink permeability, dot formability, and dry characteristics of printed images on a recording medium, such as a general paper or a special paper. The polyhydric alcohol-based or its derivative solvents are not easily evaporated and can lower a freezing point of ink, thereby improving a storage stability of the ink, and thus, prevent nozzle clogging.

With regard to the alcohol-based solvents that can be used as the organic solvent, examples of primary alcohols may include lower alcohols, such as methyl alcohol, ethyl alcohol, n-propyl alcohol, i-propyl alcohol, n-butyl alcohol, sec-butyl alcohol, t-butyl alcohol, and isobutylalcohol; ethyl alcohol, i-propyl alcohol, and n-butyl alcohol being preferred, and examples of polyhydric alcohols may include ethyleneglycol, diethyleneglycol, triethyleneglycol, propyleneglycol, butyleneglycol, 1,4-butanediol, 1,2,4-butanetriol, 1,5-pentanediol, 1,2,6-hexanetriol, hexyleneglycol, glycerol, glycerol ethoxylate, and trimethylolpropane ethoxylate. The ketone-based solvents may be acetone, methylethylketone, diethylketone, or diacetonealcohol, and the ester-based solvents may be methyl acetate, ethyl acetate, or ethyl lactate.

Examples of the nitrogen-containing solvents that can be used as the organic solvent may include 2-pyrrolidone and N-methyl-2-pyrrolidone, and examples of the sulfur-containing solvents may include dimethylsulfoxide, tetramethylenesulfone, and thioglycol.

When the organic solvent is used in combination with an aqueous solvent, such as water, the content of the organic solvent may be 0.1 to 130 parts by weight based on 100 parts by weight of the aqueous solvent. If the content of the organic solvent is less than 0.1 parts by weight, ink evaporation may occur too fast, thereby lowering stability. On the other hand, if the content of the organic solvent exceeds 130 parts by weight, the viscosity of ink may increase, thereby lowering ejection performance.

The ink composition according to the present general inventive concept may include various additives in order to reinforce the characteristics of the ink composition. In more detail, the ink composition according to the present general inventive concept may further include at least one of a wetting agent, a dispersing agent, a surfactant, a viscosity modifier, a pH adjuster, and an antioxidant. The content of the additive may be 0.1 to 20 parts by weight, preferably 0.1 to 10 parts by weight, based on 100 parts by weight of the ink composition.

The wetting agent added in the inkjet ink composition according to the present general inventive concept serves to prevent clogging of the ink composition in a nozzle, and thus, may be a polyhydric alcohol. Examples of the wetting agent include glycerine, ethylene glycol, diethylene glycol, triethylene glycol, propylene glycol, dipropylene glycol, hexylene glycol, 1,3-butanediol, 1,4-butanediol, 1,5-pentanediol, 2-butene-1,4-diol, 2-methyl-2-pentanediol, and a mixture thereof. In the ink composition according to the present general inventive concept, the content of the wetting agent may be 5 to 20 parts by weight based on 100 parts by weight of the ink composition.

The inkjet ink composition according to the present general inventive concept may further include a surfactant in order to adjust a surface tension of the ink composition, thus stabilizing the jetting performance of ink from a nozzle, and to adjust a degree of permeation of ink onto a medium. The surfactant may be an anionic surfactant, a cationic surfactant, or a nonionic surfactant. Examples of the anionic surfactant may include alkylcarboxylate salts having 1-1000 carbon atoms (preferably, alkylcarboxylate salts having 10-200 carbon atoms), salts of alkylsulfonic acid ester having 1-1000 carbon atoms (preferably, salts of alkylsulfonic acid ester having 10-200 carbon atoms), salts of alkylsulfonic acid having 1-1000 carbon atoms (preferably, salts of alkylsulfonic acid having 10-200 carbon atoms), salts of alkylbenzenesulfonic acid having 1-1000 carbon atoms (preferably, salts of alkylbenzenesulfonic acid having 10-200 carbon atoms), and mixtures thereof.

Examples of the cationic surfactant may include fatty acid amine salts, quaternary ammonium salts, sulfonium salts, phosphonium salts, and mixtures thereof. Examples of the nonionic surfactant may include polyoxyethylene alkyl ether where alkyl has 1-1000 carbon atoms, preferably, 10-200 carbon atoms), polyoxyethylene alkyl phenyl ether where alkyl has 1-1000 carbon atoms, preferably 10-200 carbon atoms), polyoxyethylene secondary alcohol ether, a polyoxyethylene-oxypropylene block copolymer, polyglycerine fatty acid ester, sorbitan fatty acid ester, and mixtures thereof. In the ink composition according to the present general inventive concept, the content of the surfactant may be 0.1 to 5 parts by weight based on 100 parts by weight of the ink composition.

In the inkjet ink composition according to the present general inventive concept, when the colorant is not a self-dispersible pigment or is a water-insoluble dye, at least one dispersing agent may be included in order to impart dispersion stability to the colorant. A dispersing agent that can be used in the present general inventive concept is not particularly limited. That is, the ink composition according to the present general inventive concept may include a high molecular weight dispersing agent (e.g., block copolymer) that may restrict physical properties, stability, and functionality of ink, in addition to a structurally simple and relatively low molecular weight dispersing agent. Examples of the structurally simple and relatively low molecular weight dispersing agent include, but are not limited to, polyvinylalcohol (PVA), cellulosics, ethylene oxide-modified phenols, ethylene oxide/propylene oxide polymers, a sodium polyacrylate solution (TEGO, disperse 715W), a modified polyacryl resin solution (TEGO, disperse 735W), a solution of an alkylol ammonium salt of a low molecular weight polycarboxylic acid polymer (BYK-Chemie, Disperbyk), an alkylol ammonium solution of a polyfunctional polymer (BYK-Chemie, Disperbyk-181), and mixtures thereof. Examples of a structurally complex and high molecular weight dispersing agent include, but are not limited to, siloxanes, such as a polyether siloxane copolymer (TEGO, Wet KL 245/Wet 260), hydrophilic polymers having a AB or BAB structure (that is, AB or BAB polymers which are hydrophilic polymers where A is a hydrophobic homopolymer or copolymer of an unsubstituted or substituted C1-C30 acrylic monomer and B is a hydrophilic polymer or copolymer of an unsubstituted or substituted C1-C30 acrylic monomer), etc. More specifically, examples of the structurally complex and high molecular weight dispersing agent include, but are not limited to, an acrylic acid/acrylate copolymer, a methacrylic acid/methacrylate copolymer, an acrylic acid/polydialkylsiloxane/acrylate block copolymer, and mixtures thereof. In the ink composition according to the present general inventive concept, the content of the dispersing agent may be 0.1 to 5 parts by weight based on 100 parts by weight of the ink composition.

The ink composition according to the present general inventive concept may further include a viscosity modifier. The viscosity modifier adjusts a viscosity of the ink composition in order to efficiently perform the spray of ink from a nozzle. Examples of the viscosity modifier include, but are not limited to, casein, hydroxymethylcellulose, hydroxyethylcellulose, carboxymethylcellulose, etc. The content of the viscosity modifier may be 0.1 to 5.0 parts by weight based on 100 parts by weight of the ink composition.

The ink composition according to the present general inventive concept may have a surface tension of 15 to 70 dyne/cm, preferably 25 to 55 dyne/cm, at 20° C., and a viscosity of 1.5 to 20 cps, preferably 1.5 to 3.5 cps, in order to assure an optimal use of the ink composition.

A method of preparing an ink composition according to an embodiment of the present general inventive concept is as follows. A colorant, a solvent, and a compound of Formula 1 are mixed, and when needed, a wetting agent, a surfactant, a dispersing agent, a viscosity modifier, etc. can be added thereto. The reaction mixture is sufficiently stirred with a stirrer to obtain a uniform solution. The resultant solution is filtered through a filter to obtain an ink composition according to an embodiment of the present general inventive concept.

The present general inventive concept also provides an ink set comprised of two or more different ink compositions according to the present general inventive concept. The ink set can be provided in an ink receiver or an inkjet cartridge of an inkjet recording apparatus. An inkjet recording apparatus according to the present general inventive concept may include a thermal head ejecting ink droplets using vapor pressure generated by heating an ink composition, a piezo head ejecting ink droplets using a piezo device, a disposable head, or a permanent head. The thermal head is particularly preferred. The inkjet recording apparatus according to the present general inventive concept may be a scanning type printer or an array type printer. An array type printer having 10,000 nozzles is preferred. The inkjet recording apparatus according to the present general inventive concept can be used for desktop, textile, and industrial purpose. The types of heads and the types and applications of printers related to the inkjet recording apparatus according to the present general inventive concept are intended to only explain the inkjet recording apparatus of the present general inventive concept, and an inkjet recording apparatus to which an ink composition according to the present general inventive concept can be applied is not limited to the above-described inkjet recording apparatus and can be selected from various inkjet recording apparatuses.

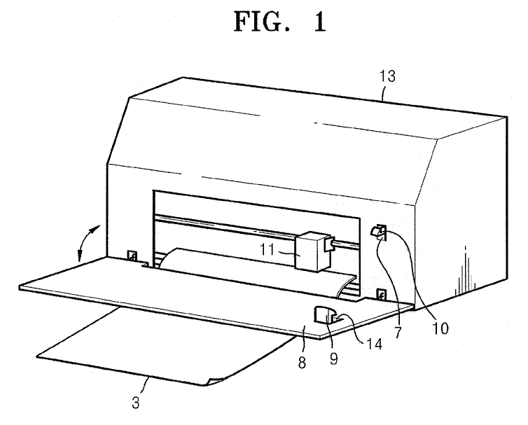

FIG. 1 is a view illustrating an inkjet recording apparatus according to an embodiment of the present general inventive concept.

Referring to FIG. 1, the inkjet recording apparatus includes an inkjet printer cartridge 11 including an ink composition containing a macro-chromophore-containing colorant and a colorant-like additive. A printer cover 8 is connected to a printer main body 13. A latching portion of a movable latch 10 is projected outward from a hole 7, and the movable latch 10 is engaged with a fixed latch 9. When the printer cover 8 is closed, the fixed latch 9 is connected to an inner portion of the printer main body 13. The printer cover 8 has a recess 14 corresponding to the latching portion of the movable latch 10 which is extended through the hole 7. The inkjet printer cartridge 11 is installed such that ink is ejected onto a paper 3 that passes through a lower portion of the inkjet printer cartridge 11.

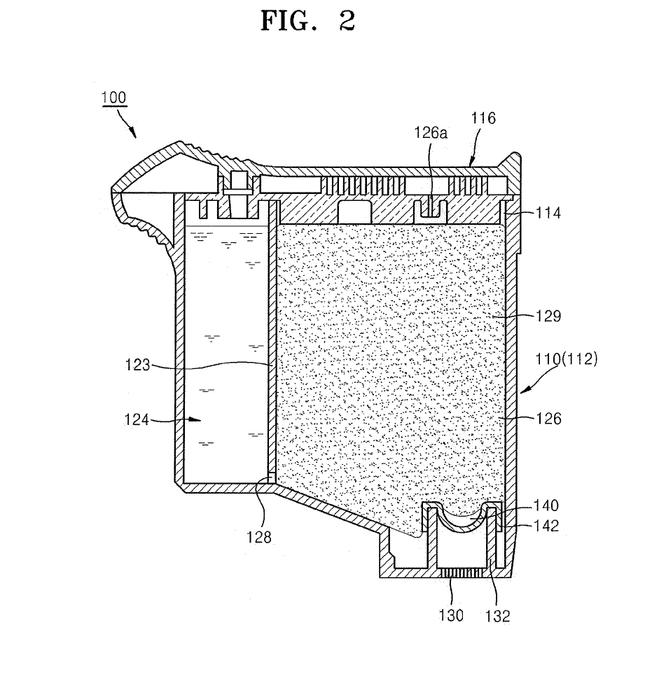

FIG. 2 is a sectional view illustrating an inkjet printer cartridge 100 including an ink set according to an embodiment of the present general inventive concept. Referring to FIG. 2, the inkjet printer cartridge 100 includes a cartridge main body 110 defining an ink reservoir 112, an inner cover 114 covering a top portion of the ink reservoir 112, and an outer cover 116 which is separated from the inner cover 114 by a predetermined distance and seals the ink reservoir 112 and the inner cover 114.

The ink reservoir 112 is divided into a first chamber 124 and a second chamber 126 by a vertical barrier wall 123. An ink passage 128 is formed at a bottom of the vertical barrier wall 123 between the first chamber 124 and the second chamber 126. Ink is filled in the first chamber 124 and in a sponge 129, and then in the second chamber 126. A vent hole 126a is formed at the inner cover 114 corresponding to the second chamber 126.

A filter 140 is disposed at a bottom side of the second chamber 126 and filters impurities and fine bubbles in the ink so as to prevent clogging of an ejection hole of a printhead 130. A hook 142 is disposed (coupled) on an edge of the filter 140 and at a top portion of a standpipe 132. Ink of the ink reservoir 112 is ejected in the form of fine droplets onto a print medium via the ejection hole of the printhead 130.

Hereinafter, the present general inventive concept will be described more specifically with reference to the following examples. The following examples are only for illustrative purposes and are not intended to limit the scope of the general inventive concept:

### Example 1

The following components were completely mixed with a mixer to prepare an inkjet ink composition.

| | |
|---|---|
| Direct Blue 199 | 4 wt % |
| Diethylene glycol | 15 wt % |
| Ethylene glycol | 5 wt % |
| Surfynol 465 | 1 wt % |
| 4-Aminophthalonitrile | 2 wt % |
| Water | 70 wt % |

### Example 2

The following components were completely mixed with a mixer to prepare an inkjet ink composition.

| | |
|---|---|
| Acid Yellow 23 | 5 wt % |
| Diethylene glycol | 12 wt % |
| Ethylene glycol | 5 wt % |
| Glycerine | 4 wt % |
| Surfynol 465 | 1 wt % |
| 2,3-Dicyanohydroquinone | 3 wt % |
| Water | 70 wt % |

### Example 3

The following components were completely mixed with a mixer to prepare an inkjet ink composition.

| | |
|---|---|
| Acid Red 289 | 4 wt % |
| Diethylene glycol | 5 wt % |
| Ethylene glycol | 10 wt % |
| Glycerine | 2 wt % |
| Surfynol 465 | 1 wt % |
| 4-Aminophthalonitrile | 3 wt % |
| Water | 75 wt % |

### Example 4

The following components were completely mixed with a mixer to prepare an inkjet ink composition.

| | |
|---|---|
| Acid Blue 9 | 3 wt % |
| 1,5-Pentanediol | 5 wt % |
| Ethylene glycol | 8 wt % |
| PEG 200 | 4 wt % |
| IPA | 2 wt % |
| Surfynol 465 | 1 wt % |
| 4-Nitrophthalonitrile | 3 wt % |
| Water | 74 wt % |

### Example 5

The following components were completely mixed with a mixer to prepare an inkjet ink composition.

| | |
|---|---|
| Cabo-jet 200 | 5 wt % |
| Diethylene glycol | 4 wt % |
| Ethylene glycol | 8 wt % |
| PEG 600 | 4 wt % |
| Surfynol 465 | 1 wt % |
| 4-Nitrophthalonitrile | 1 wt % |
| Water | 77 wt % |

### Comparative Example 1

| | |
|---|---|
| Acid Yellow 23 | 5 wt % |
| Diethylene glycol | 13 wt % |
| Ethylene glycol | 7 wt % |
| Glycerine | 4 wt % |
| Surfynol 465 | 1 wt % |
| Water | 70 wt % |

### Comparative Example 2

| | |
|---|---|
| Acid Red 289 | 4 wt % |
| Diethylene glycol | 5 wt % |
| Ethylene glycol | 12 wt % |
| Glycerine | 2 wt % |
| Surfynol 465 | 1 wt % |
| Water | 76 wt % |

### Comparative Example 3

| | |
|---|---|
| Acid Blue 9 | 3 wt % |
| 1,5-Pentanediol | 5 wt % |
| Ethylene glycol | 10 wt % |
| PEG 200 | 4 wt % |
| IPA | 2 wt % |
| Surfynol 465 | 1 wt % |
| Water | 75 wt % |

### Experimental Examples

### 1) Evaluation of Ink Storage Stability

The ink compositions prepared in Examples 1-5 and Comparative Examples 1-3 (100 ml for each) were placed in heat-resistance glass bottles. The glass bottles were sealed and incubated in a 60° C. thermostat bath for two months to determine the presence or absence of precipitates in the bottoms of the glass bottles. The evaluation was made by the following criteria and the results are presented in Table 1 below.

◯: absence of precipitates

X: presence of precipitates

### 2) Evaluation of Water Repellency

The ink compositions prepared in Examples 1-5 and Comparative Examples 1-3 were loaded in C-60 color ink cartridges and M-50 black ink cartridges (manufactured by Samsung Electronics Co., Ltd.). Then, solid boxes (▪) of 20×20 mm were printed on general papers and dried, and the optical density (OD) values of the images were measured. The images were dipped in a beaker filled with distilled water for one minute, removed, and dried, and the OD values of the images were measured. The evaluation was made by the following criteria and the results are presented in Table 1 below.

⊚: less than 15% change from initial OD value

◯15% to 30% change from initial OD value

X: 30% or more change from initial OD value

### 3) Evaluation of Clogging

The ink compositions prepared in Examples 1-5 and Comparative Examples 1-3 were loaded in C-60 and M-50 Samsung ink cartridges (manufactured by Samsung Electronics Co., Ltd.). Then, the cartridges were incubated in a state wherein nozzles were not sealed under room temperature/atmospheric pressure conditions for one month, and installed in Samsung printers (MJC-3300p). Nozzle cleaning and pattern printing were repeatedly performed. The evaluation was made by the following criteria and the results are presented in Table 1 below.

◯: removal of clogging by less than five nozzle cleaning cycles

X: no removal of clogging by less than five nozzle cleaning cycles

TABLE 1

| | Ink storage | Water | |
|---|---|---|---|
| Section | stability | repellency | Clogging |
| Example 1 | ◯ | ⊚ | ◯ |
| Example 2 | ◯ | ◯ | ◯ |
| Example 3 | ◯ | ⊚ | ◯ |
| Example 4 | ◯ | ◯ | ◯ |
| Example 5 | ◯ | ⊚ | ◯ |
| Comparative | ◯ | X | ◯ |
| Example 1 | | | |
| Comparative | ◯ | ◯ | ◯ |
| Example 2 | | | |
| Comparative | ◯ | X | ◯ |
| Example 3 | | | |

As can be seen from Table 1 above, an ink composition according to the present general inventive concept maintains ink storage stability and anti-clogging property, and at the same time, has good water repellency, thus improving durability of images.

An ink composition according to the present general inventive concept has a minimal degree of blurring of printed images with water on a general paper, does not cause nozzle clogging, and has good storage stability, and thus, can be efficiently used in inkjet printing, etc.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

What is claimed is:

1. An inkjet ink composition, comprising:
a colorant;
a solvent; and
a compound represented by Formula 1 below: 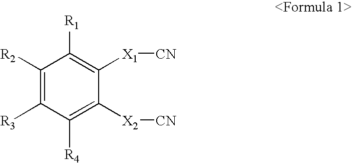
wherein:
R1, R2, R3, and R4 are each independently one of a hydrogen atom, a nitro group, an amino group, a hydroxy group, halogen, a cyano group, a carboxyl group, a substituted or unsubstituted C1-C20 alkyl group, a substituted or unsubstituted C2-C20 alkenyl group, a substituted or unsubstituted C2-C20 alkynyl group, a substituted or unsubstituted C1-C20 alkoxy group, a substituted or unsubstituted C2-C20 alkoxycarbonyl group, a substituted or unsubstituted C6-C30 aryl group, a substituted or unsubstituted C1-C20 heteroalkyl group, a substituted or unsubstituted C3-C20 heteroaryl group, a substituted or unsubstituted C2-C20 alkylcarbonyl group, a substituted or unsubstituted C7-C30 arylcarbonyl group, and a substituted or unsubstituted C4-C30 heteroarylcarbonyl group, and at least one of R1, R2, R3, and R4 comprises at least one functional group selected from a group consisting of an amino group, a nitro group, and a hydroxy group, and
X1 and X2 are each independently one of a bond, C1-C20 alkylene, C2-C20 alkenylene, C2-C20 alkynylene, C6-C30 arylene, C1-C20 heteroalkylene, and C3-C30 heteroarylene.

2. The inkjet ink composition of claim 1, wherein:
R1, R2, R3, and R4 are each independently one of a hydrogen atom, a nitro group, an amino group, a hydroxy group, a C1-C20 alkyl group, a C2-C20 alkenyl group, and a C2-C20 alkynyl group,
at least one hydrogen atom of the alkyl group, the alkenyl group, or the alkynyl group is substituted by one of a nitro group, an amino group, and a hydroxy group,
and at least one of R1, R2, R3, and R4 comprises at least one functional group selected from a group consisting of a nitro group, an amino group, and a hydroxy group.

3. The inkjet ink composition of claim 1, wherein each of X1 and X2 is a bond.

4. The inkjet ink composition of claim 1, wherein the compound of Formula 1 is a compound represented by Formula 2 below: 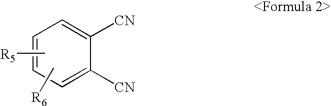
wherein:
R5 and R6 are each independently one of a nitro group, an amino group, a hydroxy group, a C1-C20 alkyl group, a C2-C20 alkenyl group, and a C2-C20 alkynyl group, and
at least one hydrogen atom of the alkyl group, the alkenyl group, or the alkynyl group is substituted by one of a nitro group, an amino group, and a hydroxy group.

5. The inkjet ink composition of claim 1, wherein the compound of Formula 1 is one of 4-aminophthalonitrile, 2,3-dicyanohydroquinone, and 4-nitrophthalonitrile.

6. The inkjet ink composition of claim 1, wherein a content of the compound of Formula 1 is 0.1 to 10 parts by weight based on 100 parts by weight of the inkjet ink composition.

7. The inkjet ink composition of claim 1, wherein the colorant is one of a dye, a pigment, a self-dispersible pigment, and a mixture thereof.

8. The inkjet ink composition of claim 1, wherein a content of the colorant is 0.1 to 15 parts by weight based on 100 parts by weight of the inkjet ink composition.

9. The inkjet ink composition of claim 1, wherein the solvent is one of an aqueous solvent, an organic solvent, and a mixture thereof.

10. The inkjet ink composition of claim 1, wherein a content of the solvent is 70 to 90 parts by weight based on 100 parts by weight of the inkjet ink composition.

11. The inkjet ink composition of claim 1, wherein the organic solvent is at least one of an alcohol-based solvent, a ketone-based solvent, an ester-based solvent, a polyhydric alcohol-based solvent, a nitrogen-containing solvent, and a sulfur-containing compound.

12. The inkjet ink composition of claim 10, wherein the solvent comprises 100 parts by weight of an aqueous solvent and 0.1 to 130 parts by weight of an organic solvent.

13. The inkjet ink composition of claim 1, wherein a surface tension of the inkjet ink composition is about 15 to 70 dyne/cm at 20° C.

14. The inkjet ink composition of claim 1, wherein a viscosity of the inkjet ink composition is about 1.5 to 20 cps.

15. A polychromatic ink set comprising two or more inkjet ink compositions, each inkjet ink composition comprising:
a colorant;
a solvent; and
a compound represented by Formula 3 below: 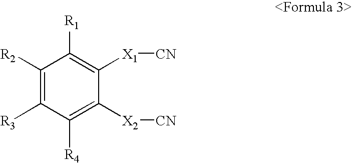
wherein:
R1, R2, R3, and R4 are each independently one of a hydrogen atom, a nitro group, an amino group, a hydroxy group, halogen, a cyano group, a carboxyl group, a substituted or unsubstituted C1-C20 alkyl group, a substituted or unsubstituted C2-C20 alkenyl group, a substituted or unsubstituted C2-C20 alkynyl group, a substituted or unsubstituted C1-C20 alkoxy group, a substituted or unsubstituted C2-C20 alkoxycarbonyl group, a substituted or unsubstituted C6-C30 aryl group, a substituted or unsubstituted C1-C20 heteroalkyl group, a substituted or unsubstituted C3-C20 heteroaryl group, a substituted or unsubstituted C2-C20 alkylcarbonyl group, a substituted or unsubstituted C7-C30 arylcarbonyl group, and a substituted or unsubstituted C4-C30 heteroarylcarbonyl group, and at least one of R1, R2, R3, and R4 comprises at least one functional group selected from a group consisting of an amino group, a nitro group, and a hydroxy group, and
X1 and X2 are each independently one of a bond, C1-C20 alkylene, C2-C20 alkenylene, C2-C20 alkynylene, C6-C30 arylene, C1-C20 heteroalkylene, and C3-C30 heteroarylene.

16. A cartridge usable in an inkjet recording apparatus, comprising the polychromatic ink set of claim 15.

17. An inkjet recording apparatus comprising the cartridge of claim 16.

18. The inkjet recording apparatus of claim 17, wherein the inkjet recording apparatus comprises a thermal head.

19. The inkjet recording apparatus of claim 17, wherein the inkjet recording apparatus comprises an array head comprising 10,000 or more nozzles.

20. An inkjet ink composition comprising:
a colorant;
a solvent; and
a crosslinker to link the colorant to a printing medium.

21. The inkjet composition of claim 20, wherein the crosslinker comprises a nitrile group to bind with the colorant, and one of a nitro group, amino group, and hydroxy group to bind with the printing medium.

22. The inkjet composition of claim 21, wherein the crosslinker is represented by Formula 4 below: 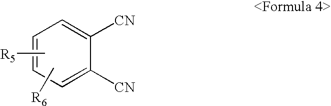
R5 and R6 are each independently one of a nitro group, an amino group, a hydroxy group, a C1-C20 alkyl group, a C2-C20 alkenyl group, and a C2-C20 alkynyl group, and
at least one hydrogen atom of the alkyl group, the alkenyl group, or the alkynyl group is substituted by one of a nitro group, an amino group, and a hydroxy group.

23. The inkjet composition of claim 21, wherein the crosslinker is one of 4-aminophthalonitrile, 2,3-dicyanohydroquinone, and 4-nitrophthalonitrile.

24. A crosslinker usable in inkjet ink composition to chemically link a colorant in the ink composition to a printing medium, the crosslinker comprising:
a nitrile group to bind with the colorant; and
one of a nitro group, amino group, and hydroxy group to bind with the printing medium.

25. The crosslinker of claim 24, wherein the crosslinker is a dicyanobenzene-based compound.
